(19) United States
(12) Patent Application Publication
(10) Pub. No.: US 2003/0073961 A1
(43) Pub. Date: Apr. 17, 2003

(54) MEDICAL DEVICE CONTAINING LIGHT-PROTECTED THERAPEUTIC AGENT AND A METHOD FOR FABRICATING THEREOF

(76) Inventor: Dorrie M. Happ, San Jose, CA (US)

Correspondence Address: Squire, Sanders & Dempsey L.L.P., Suite 300, One Maritime Plaza, San Francisco, CA, 94111, US

(21) Appl. No.: 09/966,036

(22) Filed: Sep. 28, 2001

(51) Int. Cl. A61M 5/32; A61K 31/337; A61K 9/14

(52) U.S. Cl. 604/274; 424/486; 514/449

(57) ABSTRACT

Light- and/or UV-radiation protective coatings for drug delivery devices, such as, for instance, drug eluting vascular stents, where the drugs being delivered via the stents are light sensitive. A method of fabricating a medical article, such as a drug eluting vascular stent, that includes the light- and/or UV-radiation protective coating.

### BACKGROUND OF THE INVENTION

1. Field of the Invention.

This invention relates to the field of medical devices, especially those used for delivery of drugs. More particularly, it is directed to light protective coating compositions for drug delivery devices, such as, for instance, drug eluting vascular stents, where the drugs being delivered via the stents are light sensitive.

2. Description of Related Art.

In the field of medicine, there is frequently a necessity to administer drugs to the patients locally. Such localized drug delivery is often a method of treatment preferred by physicians because, due to the delivery in a precise diseased site, overall smaller doses of the medicine would be required vis-a-vis other methods of drug delivery. Therefore, side effects associated with local delivery are less frequent compared with the side effects associated with other methods of drug delivery and the efficacy of treatment is generally improved.

Stents are being treated so as to provide a vehicle for local drug delivery. The medicine to be administered can be released through the stent in a variety of ways, for example, by a polymeric coating deposited on the stent. The coating, in addition, can have other important functions, such as providing the stent with increased lubricity and serve as an oxygen and/or water vapor barrier.

Currently, a typical embodiment used to achieve local drug delivery via stent comprises a stent coated with a three-layer composition shown on FIG. 1 and described subsequently. The three layer composition includes a drug-polymer layer 3, a primer polymer layer 2 for improving adhesion of the drug-polymer layer 3, and a topcoat polymer layer 4 providing rate limiting barrier, lubricity and other useful properties. The medicine to be administered according to this embodiment slowly seeps from the drug-polymer layer through the topcoat polymer layer to the diseased site in the patient's body where the stent is implanted.

However, such traditional composition has some drawbacks and disadvantages. One of the drawbacks and disadvantages is the fact that some of the drugs, which are currently being tested in the market, such as actinomycin-D, are very light sensitive and their therapeutic utility can be severely compromised, or even destroyed if they are exposed to light. Since the topcoat polymer layer is usually clear enough to allow light to pass through, the light-sensitive drug in the drug-polymer layer often needs special protection.

In order to protect the drug in the drug-polymer layer, the manufacturing of the coated stent must take place in the environment with filtered light, where the wavelengths which can negatively affect the drug have been filtered out.

Even though light sensitivity of some drugs (for example, that of actinomycin-D), when the drug has already been incorporated into the stent, is not as high as during the manufacturing process, other drugs might be equally light-sensitive either during the process of manufacturing of the stent or afterwards, in the finished stent.

Therefore, it is still advisable, for drugs that are at least as light-sensitive as actinomycin-D, that post-processing steps should also be performed under filtered light. These steps commonly include crimping, inspecting, packaging and the like, as well as handling the stent in the field.

In view of the foregoing, there is a need to prepare a composition for the stent where the drug is light-protected, since using filtered light as described above is cumbersome, inconvenient and expensive. This need remains unmet.

Consequently, it is very desirable to prepare a polymeric coating for medicated stents which includes a component for protecting against light and/or UV-radiation. Such coatings are unknown in the art.

References do teach compositions utilizing light-protective coatings for variety of application. For instance, U.S. Pat. No. 5,900,425 to Kanikanti, et. al. discloses pharmaceutical preparations having controlled release of the active compound. These preparations are typically administered orally. If the active compound is light-sensitive (Kanikanti, et. al. disclose nifedipine and nimodipine), the controlled-release tablets are provided with a light-protective coating in order to preserve the light-sensitive medicine from degradation.

As an example, Kanikanti, et. al. recommend spraying a water-based suspension of a film former, PEG (plasticizer), titanium dioxide and iron oxide (the light-scattering and absorbing pigments), followed by drying in hot air. Obviously, Kanikanti, et. al. use TiO₂ and Fe₂O₃ as light-protective compounds. However, Kanikanti, et. al. deal exclusively with tablets for oral administration. This reference does not describe nor suggest using light-protective compounds on stents. The difference in applications is quite substantial. In fact, a light protective coating for an oral tablet is fundamentally different than a light protective coating for an implantable device.

Using materials such as Fe₂O₃ to protect against light may be acceptable in the light protective coating for an oral tablet, but is not an acceptable method for the stent coatings because the stent coatings must be extremely inert and must not interfere with the body's inflammatory response in any way. Some experts have theorized that the etiology of restenosis is caused by inflammatory response. Materials ingested orally and which are subsequently excreted can be much more toxic than a material that is implanted in the tissues. In addition, the method described by Kanikanti, et. al. suggest using hot air to dry the light protective compound. In many cases the drug may be heat sensitive and cannot tolerate drying conditions at high temperatures. Moreover, for the tablets described by Kanikanti, et. al. there is no issue of post-processing raised by the inventors.

Clearly, the only protection from light that the tablets require in Kanikanti, et. al. is during storage. This protection can be easily achieved in a variety of ways, for instance, by using dark-glass tablets containers. Therefore, using the light protective layer containing titanium and iron oxides is truly optional. These alternative approaches cannot be used for stent coatings since the drug needs the most protection from light during the manufacturing process and post-processing when degradation is most likely to occur.

In another reference, U.S. Pat. No. 5,314,741 to Roberts, et. al., a polymeric article (a rubber article) is disclosed which is coated with a thin layer of a coating resistant to light and other elements (i.e., oxygen or ozone). Roberts, et. al. apply the light-protective coating on a polymeric substrate requiring protection. This substrate is rubber or a similar vulcanized diene-derived elastomer. It is well known to those skilled in the art that such elastomers are highly vulnerable to UV radiation and oxidants and degrade easily unless special steps are taken to protect them.

Yet another patent, U.S. Pat. No. 5,756,793 to Valet, et. al. describes a method of protecting surfaces of wood against damage by light and a protective coating for wood. Surfaces of wood which are exposed to intense sunlight are damaged primarily by the UV component of sunlight. The polymeric constituents of the wood are degraded as a consequence, leading to a roughening and discoloration of the surface.

The usual method of protecting wood against damage by light without giving up the visual image of the wood surface to use a colorless polymer coating containing a light stabilizer, in particular a UV absorber. Valet, et. al. teach the use of a derivative of benzophenone as an UV absorber. Such compounds display a distinct stabilizer action against the effect of light, when applied in a coating composition.

Both Roberts, et. al. and Valet, et. al., however, disclose only compositions where it is the outer surface of the substrate, be it rubber or wood, that is light-protected. These references do not teach the protection of the internal layers of the composition nor the protection of any light vulnerable fillers.

In addition, these references discuss protection solely from UV-radiation. The references do not describe a material having properties allowing for the protection of a light-sensitive drug, more specifically, a drug in an implantable device, where the protection is provided from both UV and/or visible light degradation. Yet a need to have such material is acute.

The present invention provides a number of such lightand/or UV-radiation protected coatings for implantable devices such as stents according to the following description.

### SUMMARY OF THE INVENTION

This invention provides a light-protected polymer coating for medical devices, particularly, for medicated stents containing light-sensitive drugs.

The coating comprises a coating applied on the surface of the stent. The coating according to embodiments of this invention optionally includes a polymer primer layer applied directly on the surface of the stent, a drug-polymer layer disposed on top of the primer polymer layer, and optionally a topcoat polymer layer applied on top of the drug-polymer layer.

The coating includes a light-sensitive drug. In order to protect this drug from light and/or UV-radiation, a light- and/or UV-radiation protective compound is included in the coating.

In one embodiment of this invention, the light- and/or UV-radiation protective compound is added to the topcoat polymer layer and so filled topcoat polymer layer is applied on top of the drug-polymer layer, instead of the pure topcoat polymer layer.

In another embodiment of this invention, the light- and/or UV-radiation protective compound is added to a separate polymer layer that is applied directly on the surface of the previously applied topcoat polymer layer.

In yet another embodiment, the light- and/or UV-radiation protective compound is added directly to the drug-polymer layer. This embodiment can be also combined with the other two embodiment discussed above.

In any of the embodiments, the drug of the drug-polymer layer is protected from the light-and/or UV-radiation-induced deterioration, degradation and destruction, thus ensuring the preservation of the therapeutical properties of the drug when it is incorporated in the stent.

According to one aspect of this invention, a coating for medical devices is provided, the coating having increased light resistance, the coating comprising a drug-polymer layer containing a drug included into the drug-polymer layer, and a light- and/or UV-protective compound incorporated into the coating.

According to another aspect of this invention, a coating for medical devices is provided, the coating having increased light resistance properties, the coating comprising a drug-polymer layer containing a drug incorporated into the drug-polymer layer, and a topcoat polymer layer, where a light- and/or UV-protective compound dispersed within the topcoat layer.

According to yet another aspect of this invention, a coating for medical devices is provided, the coating having increased light resistance properties and including a drug-polymer layer and a topcoat layer, where a film-forming polymer layer disposed upon the topcoat layer, and the light- and/or UV-protective compound is dispersed in the film-forming polymer.

According to another aspect of this invention, a coating for medical devices is provided, the coating having increased light resistance properties and including a drug-polymer layer, where light- and/or UV-protective compound is dispersed within the drug-polymer layer.

According to yet another aspect of this invention, a method for fabricating a medical article is provided, the method comprising providing a medical device, applying a coating composition onto the medical device, wherein the coating composition has increased light resistance, such increased light resistance provided by a light- and/or UV-protective compound incorporated into the coating composition.

### BRIEF DESCRIPTION OF THE DRAWINGS

The features and advantages of the present invention will become better understood with regard to the following description, appended claims, and accompanying drawings where:

FIG. 1 schematically depicts a cross-section of a known and currently used multi-layered polymeric coating for stents.

FIG. 2A schematically depicts a cross-section of a first embodiment of multi-layered polymeric coating composition for stents of this invention.

FIG. 2B schematically depicts a cross-section of a second embodiment of multi-layered polymeric coating composition for stents of this invention.

FIG. 2C schematically depicts a cross-section of a third embodiment of multi-layered polymeric coating composition for stents of this invention.

FIG. 2D schematically depicts a cross-section of an embodiment of this invention combining the features of the embodiments depicted in FIG. 2A and FIG. 2C.

FIG. 2E schematically depicts a cross-section of an embodiment of this invention combining the features of the embodiments depicted in FIG. 2B and FIG. 2C.

### DETAILED DESCRIPTION OF THE EMBODIMENTS OF THE INVENTION

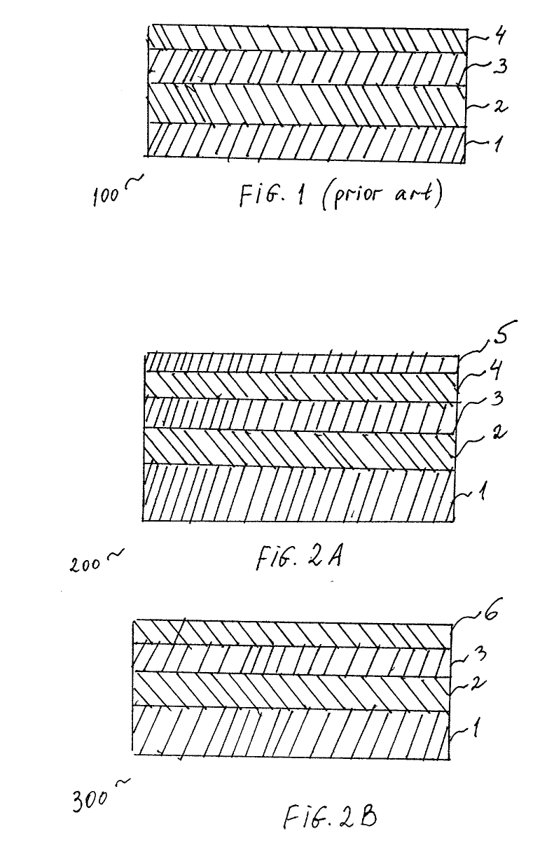

FIG. 1 shows a cross-section of a typical medical device 100 incorporating a polymer coating. This coating is currently known and used on medical devices, particularly, on stents. According to this embodiment, a stent 1 is coated with a primer polymer coating layer 2 and by a drug-polymer layer 3. The drug-polymer layer 3 comprises a polymer binder and a drug, dispersed in the binder, to be administered via the stent 1. Finally, a polymer topcoat layer 4 is applied on top of the drug-polymer layer 3 for controlling the rate of release of the drug.

As mentioned previously, the prior art system 100, shown on FIG. 1, allows for light rays to penetrate the topcoat layer 4 because this layer is typically clear and/or light-transparent. Consequently, the light reaches to the drug-polymer layer 3 and damages the drug, should the drug be light-sensitive. In fact, many of the drugs used with stents are light-sensitive.

Therefore, the system 100 is not sufficiently effective in that it does not provide light protection for the drugs contained by the drug-polymer layer 3. As a result, the drug is damaged by light and may degrade or otherwise lose its medicinal and therapeutic effectiveness. In view of this, an improved coating for providing the light protection to light sensitive drugs is highly desirable.

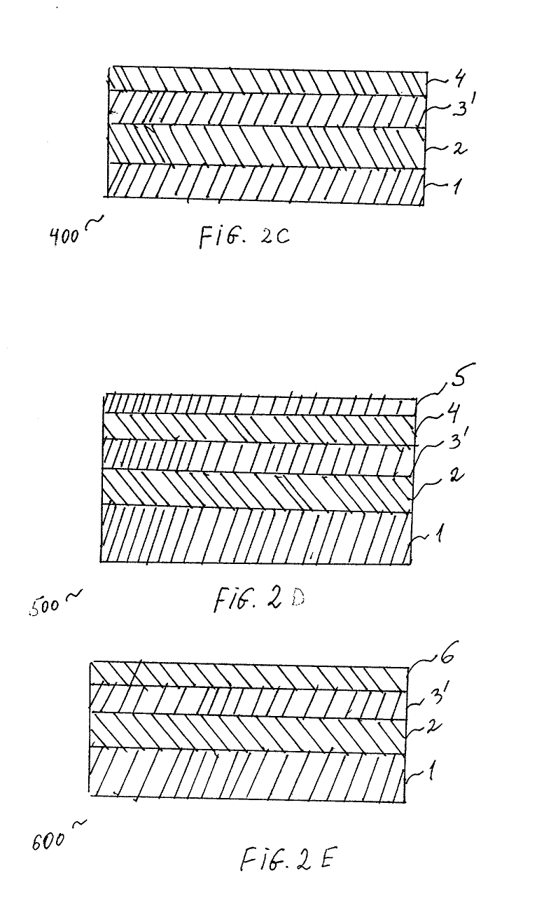

FIGS. 2A, 2B, and 2C schematically depict cross-sections of three embodiments of such an improved coating. A typical substrate on which the coating is applied is a medicated stent, for instance, a TETRA or a PIXEL stent available from Guidant Corporation. The substrate usable for this invention need not be one of the above-mentioned stents. It can be another implantable medical device. Examples of such implantable devices include stent-grafts, grafts (e.g., aortic grafts), artificial heart valves, cerebrospinal fluid shunts, pacemaker electrodes, axius coronary shunts and endocardial leads (e.g., FINELINE and ENDOTAK, available from Guidant Corporation). The underlying structure of the device can be of virtually any design. The device can be made of a metallic material or an alloy such as, but not limited to, cobalt chromium alloy (ELGILOY), stainless steel (316L), “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co. of Jenkintown, Pennsylvania. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

The first embodiment 200 is shown in FIG. 2A. It is similar to the prior art embodiment of FIG. 1 but an extra light-protective polymer layer 5 is applied on top of the topcoat polymer layer 4. The polymer in the layer 5 is typically one of the polymers commonly used for making topcoats. The layer 5 includes an compound which makes the layer 5 non-transparent. The use of the primer layer 2 in this and every other embodiment of this invention is optional. If a drug to be protected is predominantly sensitive in the UV-area, then known UV-absorbing compounds can be used, and if the sensitivity of the drug is chiefly in the visible range of wavelengths, then the compounds absorbing radiation in the visible area of the spectrum are used.

Typically, many important drugs are sensitive to radiation in both UV- and visible portions of the spectrum, and the drug-polymer layer can contain between about 5% and about 50% of the drug, by the mass of the drug-polymer layer 3.

Therefore, a compound to be used should provide protection from both UV-radiation and visible light. In addition, the compound should be compatible with the polymer in the drug-polymer layer 3 and compatible with the drug. Furthermore, the compound should be biologically compatible, so that when the device is implanted in a body, the compound will not produce any adverse responses. One of such compounds can be carbon black.

Instead of carbon black, other compounds can be also used in the alternative, as long as the compounds block visible and/or UV light and are also biocompatible with the body, drug-compatible and polymer-compatible. An example of such possible alternative compound can be gold or titanium-nitride-oxide. The necessary amount of the compound, so as to provide the proper degree of the light protection can be calculated by commonly used methods known to those having ordinary skills in the art.

The thickness of the protective layer 5 can be within a range of between about 100 nanometers and about 4 micrometers, alternatively, within a range of between about 1 micrometer and about 2 micrometers.

In another embodiment 300 of this invention shown by FIG. 2B, no separate light-protective layer is used. Instead, a lightand/or UV-radiation protective compound is added to the topcoat polymer layer 4 to form a topcoat polymer layer 6 which not only serves as a rate reducing membrane but also serves as a light-protective layer. In addition, the light- and/or UV-radiation protective compound can also serve as a means of controlling the rate of drug release. Just as for the embodiment 200 shown on FIG. 2A and described above, the compound to be used should provide protection from both UV-radiation and visible light. Again, carbon black or an alternative compound can be used.

The light- and/or UV-radiation protective compound should be biocompatible and inert to the drug of the drug-polymer layer 3. optionally, the compound may also have a therapeutic effect such as reducing platelet adhesion and fibrinogen binding. In addition to a colorant, other light- and/or UV-radiation protective compounds can be selected by those ordinarily skilled in the, taking into account the functions and the amount of the drug, as well as the above-mentioned requirements of UV- and light-protection, biocompatibility and inertness.

The amount of solids in the layer 6 (the compound plus the polymer) can be between about 0.25% (mass) and about 20% (mass) of the solution to be applied to form the layer 6. Alternatively, the amount of solids can be between 1% (mass) and about 8% (mass). The ratio, by mass, of the light- and/or UV-radiation protective compound to the polymer is between about 3 to 1 (at the lower range of concentrations of the solution to be sprayed) and about 1 to 3 (at the higher range).

The thickness of the layer 6 can be within a range of between about 100 nanometers and about 4 micrometers, alternatively, between about 1 micrometer and about 2 micrometers.

In another embodiment 400 of this invention shown by FIG. 2C, the light- and/or UV-radiation protective compound is added to the drug-polymer layer 3′. The compound is added to a solution containing the drug and the polymer component of the drug-polymer layer 3′ and the solution is applied onto the stent. This embodiment provides an additional advantage of shielding the UV- and/or light-sensitive drug during the process of applying the drug on the stent. Since the drug-containing solution is applied onto the stent before the top coat layer 4, applying the light-protective compound together with the drug would allow protection of the drug from light at an earlier step, which simplifies the manufacturing process.

For the embodiment 400 shown by FIG. 2C, the same solids contents is typically used as the solids contents described above for the embodiment 300 shown by FIG. 2B (where the compound is added to the topcoat 6). Therefore, the solids contents for the embodiment 400 of FIG. 2C (the sum of the drug, the polymer and the light- and/or UV-radiation protective compound) can be between about 0.25% (mass) and about 20% (mass) of the solution to be applied, alternatively, between 1% (mass) and about 8% (mass). The ratio, by mass, of the drug to the light- and/or UV-radiation protective compound to the polymer can be between about 1 to 1 to 2 and about 1 to 3 to 20.

In addition, for even better light and UV-radiation protection, two further embodiments, 500 and 600, shown by FIGS. 2D and 2E, respectively, can be used. Both are the hybrid embodiments. The embodiment 500 combines the features of embodiment 200 (having a separate light- and/or UV-radiation protective polymer layer 5 applied onto the topcoat 4) with the features of the embodiment 2C (having a drug-polymer layer 3′ containing the light- and/or UV-radiation protective compound). The embodiment 600 combines the features of the embodiment 300 (having the topcoat 6 with the light- and/or UV-radiation protective compound incorporated therein) also with the features of the embodiment 2C (having a drug-polymer layer 3′ containing the light- and/or UV-radiation protective compound).

In the embodiment depicted on FIG. 2C using the topcoat layer 4 is optional, and the coating can remain viable when the drug-polymer layer 3′ is the outermost layer. Furthermore, as mentioned previously, the use of the primer layer 2 is also optional. Therefore, the device of this invention can comprise just an implantable medical device coated with a drug-polymer coating containing a light- and/or UV-radiation protective compound. As another alternative, the device of this invention can comprise just an implantable medical device coated with a primer layer, on top of which the drug is applied without polymer, followed by a light- and/or radiation protective topcoat.

Either embodiment shown by FIGS. 2A, 2B or 2C can be used with any kind of the primer polymer layer 2, which would be otherwise usable, according to the criteria known to those having ordinary skill in the art. The thickness of the primer polymer layer 2 is not affected by the use of a protective layer of this invention and the method of application of the primer layer 2 remains the same.

The polymers used in either the embodiment of FIGS. 2A, 2B, and 2C, i.e., the drug-polymer layer 3, the topcoat layer 4, the protective layer 5, and the topcoat/protective polymer layer 6 are chosen according to the criteria known to those having ordinary skill in the art and as required by parameters such as the type of the device, the material of which the device is made, the type of process employed to form the coating, and a like.

Examples of polymers that can be used in the top coat layer 4, or the topcoat/protective layer 6 include ethylene-vinyl alcohol copolymer (commonly known by the generic name EVOH or by the trade name EVAL as distributed by the Aldrich Chemical Co. of Milwaukee, Wis.), poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid, PLA), poly(glycolic acid-co-trimethylene carbonate), polyphosphoester, polyphosphoester urethane, poly(amino acids), polycyanoacrylates, poly(trimethylene carbonate), poly(iminocarbonate), copoly(ether-esters) (e.g., polyethyleneoxide, PEO with PLA), polyalkylene oxalates, polyphosphazenes, biomolecules, such as fibrin, fibrinogen, cellulose, starch, collagen and hyaluronic acid, polyurethanes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene fluoride and polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

The drugs forming a part of the drug-polymer layer 3 are light-sensitive or UV-sensitive drugs, or both. Examples of such drugs include, for instance, actymicin D, paclitaxel, vincristine or other light or UV-sensitive drugs.

In every embodiment of this invention, each layer is applied by any appropriate method known to those ordinarily skilled in the art, for example, by spraying, or, alternatively, by dipping.

Having described the invention in connection with several embodiments thereof, modification will now suggest itself to those having ordinary skill in the art. As such, the invention is not to be limited to the described embodiments

What is claimed is:

1. A coating for a medical device, said coating having increased resistance to light and/or UV-radiation, said coating comprising:
(a) a drug-polymer layer containing a drug included in said drug-polymer layer; and
(b) a light- and/or UV-protective compound included in said coating.

2. The coating as claimed in claim 1, wherein said medical device is a stent.

3. The coating as claimed in claim 1, wherein said drug is a light-sensitive drug or a UV-radiation sensitive drug.

4. The coating as claimed in claim 3, wherein said light-sensitive drug comprises actymicin D, paclitaxel, or vincristine.

5. The coating as claimed in claim 1, further comprising a topcoat layer disposed upon said drug-polymer layer.

6. The coating as claimed in claim 5, wherein said light- and/or UV-protective compound is dispersed within said topcoat layer.

7. The coating as claimed in claim 6, wherein said light- and/or UV-protective compound is further dispersed within said drug-polymer layer.

8. The coating as claimed in claim 5, further comprising a film-forming polymer layer disposed on said topcoat layer, wherein said light- and/or UV-protective compound is dispersed in said film-forming polymer layer.

9. The coating as claimed in claim 1, wherein said light- and/or UV-protective compound is dispersed within said drugpolymer layer.

10. The coating as claimed in claim 1, further comprising a primer polymer layer deposited between a surface of said medical device and said drug-polymer layer.

11. The coating as claimed in claim 1, wherein said light- and/or UV-protective compound comprises carbon black or gold.

12. A method for fabricating a medical article, the method comprising forming a coating onto said medical device, wherein said coating includes light- and/or UV-protective substance.

13. A medical device comprising a coating, said coating produced according to the method of claim 12.

14. The method as claimed in claim 12, wherein said medical device is a stent.

15. The method as claimed in claim 12, wherein said coating comprises a drug-polymer layer containing a drug included into said drug-polymer layer, wherein said light- and/or UV-protective substance is incorporated into said coating.

16. The method as claimed in claim 15, wherein said drug is a light-sensitive drug or a UV-radiation sensitive drug.

17. The method as claimed in claim 16, wherein said light-sensitive drug comprises actymicin D, paclitaxel, or vincristine.

18. The method as claimed in claim 15, further comprising a topcoat layer disposed upon said drug-polymer layer.

19. The method as claimed in claim 18, further comprising a film-forming polymer layer disposed upon said topcoat layer, wherein said light- and/or UV-protective substance is dispersed in said film-forming polymer.

20. The method as claimed in claim 18, wherein said light- and/or UV-protective substance is dispersed within said topcoat layer.

21. The method as claimed in claim 20, wherein said light- and/or UV-protective substance is further dispersed within said drug-polymer layer.

22. The method as claimed in claim 15, wherein said light- and/or UV-protective substance is dispersed within said drug-polymer layer.

23. The method as claimed in claim 15, further comprising a primer polymer layer deposited between a surface of said medical device and said drug-polymer.

24. The method as claimed in claim 15, wherein said light- and/or UV-protective substance comprises carbon black or gold.
